(19) United States
(12) Patent Application Publication
(10) Pub. No.: US 2019/0147987 A1
(43) Pub. Date: May 16, 2019

(54) APPARATUS AND SYSTEM FOR LABORATORY BLOOD SAMPLE ORDERING, PROCESSING, AND COLLECTION

(71) Applicant: BRADLEY BANERIAN, BLOOMFIELD HILLS, MI (US)

(72) Inventor: BRADLEY BANERIAN, BLOOMFIELD HILLS, MI (US)

(21) Appl. No.: 16/164,339

(22) Filed: Oct. 18, 2018

Related U.S. Application Data
(60) Provisional application No. 62/547,541, filed on Aug. 18, 2017.

(51) Int. Cl. G16H 10/40; G06Q 10/10

(52) U.S. Cl. CPC G16H 10/40; G06Q 10/1093

(57) ABSTRACT

An apparatus and system for laboratory blood sample ordering, collection, and processing is described. The apparatus and system include a central computer processor, a provider user interface connected to the central computer processor, a test profile database connected to the central computer processor, and a schedule database connected to the central computer processor. An order for a blood laboratory test generates collection parameters based upon test type and the time a result is required. The system generates collection parameters, including a sample reserve fraction sufficient to provide sample for predicted future tests. Test results are communicated to the requesting provider and entered into the patient's electronic medical record. Additional sample collection is ordered if the sample reserve fraction is insufficient for subsequent requested tests.

This application claims the benefit of U.S. Provisional Application 62/547,541 filed on Aug. 18, 2017, which is incorporated herein by reference in its entirety.

No federal research funds were used in the development of this invention

### BACKGROUND

This invention relates to phlebotomy, and more specifically to an apparatus and system for the collection, storage, and processing of tissue samples used in laboratory testing, such as in a health center setting.

Phlebotomy is possibly the oldest medical procedure in history, with the earliest known examples dating back thousands of years to ancient Egypt. Today, phlebotomy is the most common invasive medical procedure, one which the College of American Pathologists estimates occurs roughly 1 billion times per year in the United States. As a result of both the frequency of phlebotomy procedures and their inherently invasive nature, the procedures represent a major challenge to the health care system.

One challenge is the inherent risk of patient and health care worker morbidity and mortality caused by complications and the transmission of pathogens. Needle stick injuries account for up to 80 percent of accidental exposure to blood, and it has been estimated that there are 800,000 accidental needle stick injuries per year in the United States. The true incidence could be much higher, as it is estimated that 96% of all accidental needle stick injuries go unreported. Improved needle technology has reduced the risk of needle stick injuries to some extent, but they continue to be a pervasive health risk among patients, phlebotomists, and healthcare professionals who draw blood for laboratory tests. The risk of needle stick injuries increases as more blood samples are drawn for testing purposes.

There are more than 20 known pathogens reportedly transmitted from accidental needle sticks. The most common is the Hepatitis B Virus (HBV). The Center for Disease Control (CDC) estimates that HBV infects 8,700 healthcare workers annually. Of those infected with HBV, 400-440 require hospitalization and an estimated 200 die each year as a result of the virus. The Hepatitis C Virus (HCV), for which there is no vaccination available, accounted for an estimated 560 to 1,120 infections in health care workers in 1995. An estimated 2% of the 800,000 accidental needle sticks per year (16,000) are likely to be contaminated by HIV.

A second challenge associated with phlebotomy procedures is the discomfort and anxiety caused by unnecessary procedures. Patient comfort and autonomy is a priority at every hospital, as hospitals continue to take steps to increase patient satisfaction at their facilities. Repeated and unnecessary blood draws tend to reduce patient satisfaction.

A third challenge associated with phlebotomy procedures is the inefficiency and unnecessary expense associated with excessive sample collections. As hospitals seek to improve cost effectiveness of services, one avenue that holds potential is through the reduction of unnecessary phlebotomy procedures. A reduction in phlebotomy procedures would reduce costs for the hospital in terms of staff and supplies needed for the blood draw process. Supplies used during the blood draw process can include safety needles, butterfly needles, syringes, sample collection tubes, tourniquets, antiseptics, gauze, puncture resistant disposal containers, and bandages. With regards to staffing cost, the United States Department of Labor estimates that the average annual income of a phlebotomist is $30,150 (USD). Inefficient use of phlebotomy and laboratory staff associated with laboratory blood tests is a significant contributor to rising healthcare costs.

A fourth challenge associated with phlebotomy procedures is that multiple venipuncture procedures increases the risk of serious medical complications to patients. The more needle sticks a patient receives, the more difficult it is to perform subsequent successful venipunctures. Repeated needle sticks can often lead to patient complications, such as phlebitis, nerve and tendon damage, hematoma formation, and bruising.

In light of these issues, it is notable that no apparatus and system has been described that is satisfactory to compile and assess provider's requests for blood test data, schedule phlebotomy procedures to reconcile the schedules of patients and health care staff, determine appropriate blood sample volumes, store a fraction of blood samples for predicted and/or additional tests, and thus reduce the cost and health risks associated with multiple unnecessary venipuncture procedures. Such an apparatus and system also could realize similar reductions in cost and health risks for other tissue collection activities.

It is desirable to implement an apparatus and system that will reduce the number of venipuncture procedures required for a patient. Such a system would allow health centers, hospitals, and other healthcare providers to reduce the number of needle sticks per patient per day, relieving the burden for patients and hospitals alike. Being the most common invasive procedure performed in hospitals, reducing venipunctures will improve patient care, cut hospital costs, and reduce instances of phlebotomy related injuries to patients and healthcare workers. One or more of these advantages may be achieved by collecting information from multiple healthcare providers regarding the need for laboratory test results and the schedule for those results, predicting additional test results that may be required based upon the tests ordered and patient diagnosis, scheduling a venipuncture procedure to satisfy various parameters, including the schedules of the requesting healthcare provider and the patient, collecting sufficient blood in one venipuncture procedure to accomplish the ordered and predicted test procedures, and facilitating the laboratory storage of blood so that follow-up tests can be performed without additional venipuncture procedures.

### SUMMARY OF THE INVENTION

A system for collection and processing of laboratory blood tests is described. A central computer processer is operably connected to a provider user interface, a test profile database, and a schedule database. In some embodiments the central computer processor is operably connected to a patient's electronic medical record (EMR). The central computer process generates blood draw parameters including time and amount of blood. The central computer processor may predict additional blood test requirements, and reserve a fraction of the blood sample for storage for later tests. The central computer processor may distribute test results in the EMR.

A system for collection and processing of laboratory tests is described with a central computer processor, a provider user interface operably connected to said computer processor, a test profile database operably connected to said computer processor; a schedule database operably connected to said computer processor; and a blood fraction storage facility. The central computer processor is adapted to receive test orders and other data, including the earliest time blood may be used and the latest time that the provider requires laboratory results from a sample, from the provider user database, test profiles from the test profile database, and patient and provider schedules from the schedule database. The central the computer processor is adapted to identify test parameters and order a test sample, including a sample reserve fraction. A test center receives a sample including a reserve sample fraction collected according to the test parameters. The sample reserve fraction is stored for subsequent predicted tests.

These and other features of the disclosed examples can be understood from the following description and the accompanying drawings, which can be briefly described as follows.

### BRIEF DESCRIPTION OF THE DRAWINGS

FIG. 1 is a diagram of the ordering phase of one embodiment of the invention.

FIG. 2 is a diagram of the processing phase of one embodiment of the invention.

FIG. 3 is a diagram of the collection phase of one embodiment of the invention.

FIG. 4 is a diagram of an alternate embodiment of the invention.

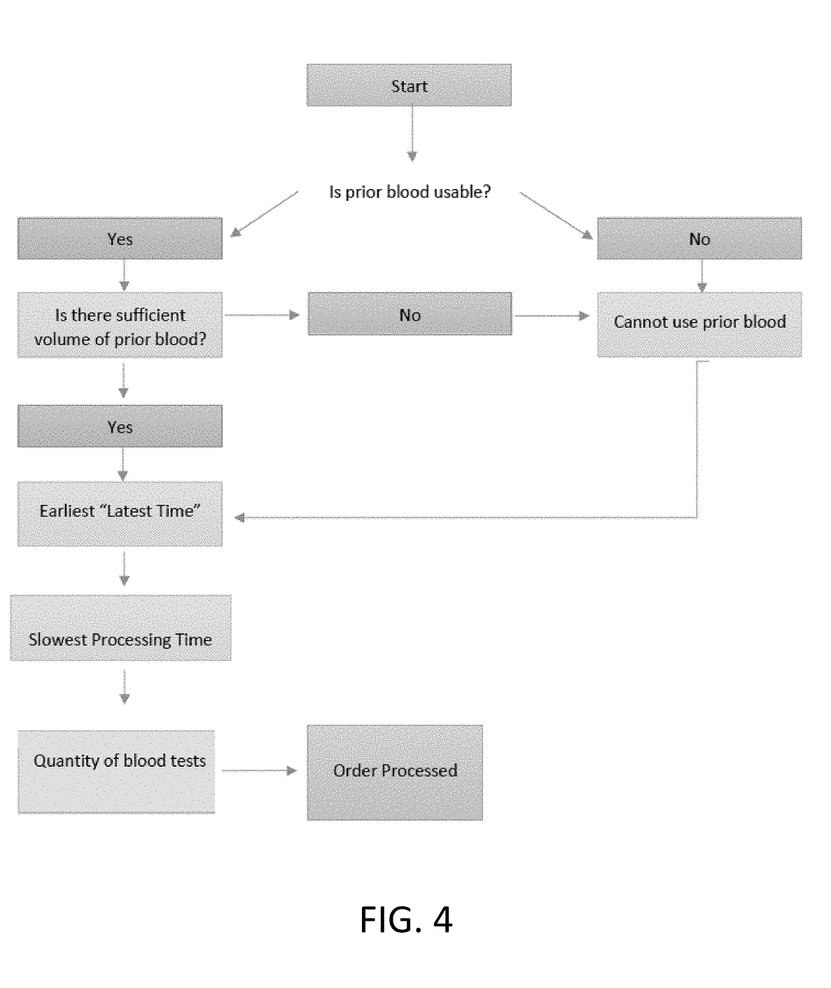

### DETAILED DESCRIPTION

The following detailed description is made with reference to the figures.

An apparatus and system for collecting, storing, and processing laboratory tissue tests is described. The apparatus and system can be implemented for the collecting, storing, and processing of blood samples. The system is generally suitable for any healthcare clinic or facility, but is particularly useful in medium to large hospitals and medical centers. The apparatus and system can be implemented in a system of multiple hospitals and medical centers with a central laboratory function.

The system and apparatus includes a provider user interface. The provider user interface allows the physician, nurse, or other healthcare provider to order a laboratory test requiring a blood sample. Blood samples are used for a vast variety of laboratory tests. Common tests include glucose tolerance, blood glucose tests, and serum lipids (triglycerides, cholesterol, and related substances.

Some types of lab tests determine whether a patient's values fall within normal ranges. Normal test values are usually given as a range, rather than as a specific number, because normal values vary from person to person. What is normal for one person may not be normal for another person.

Other types show whether there is a particular substance present or absent, such as a mutation in a gene, or an infectious organism, which indicates whether the patient has a disease, an infection, or may or may not respond to a therapy.

Some laboratory tests are precise, reliable indicators of specific health problems, while others provide more general information that gives doctors clues to possible health problems. Information obtained from laboratory tests may help doctors decide whether other tests or procedures are needed to make a diagnosis or to develop or revise a previous treatment plan. Some tests when positive often require one or more specific follow-up tests to then be performed.

The provider user interface may be a computer, a client on a network including the central computer processor, or a networked device, such as a tablet or mobile phone with suitable software to collect and share data with the central computer processor. The provider user interface may include prompts to query the provider for information.

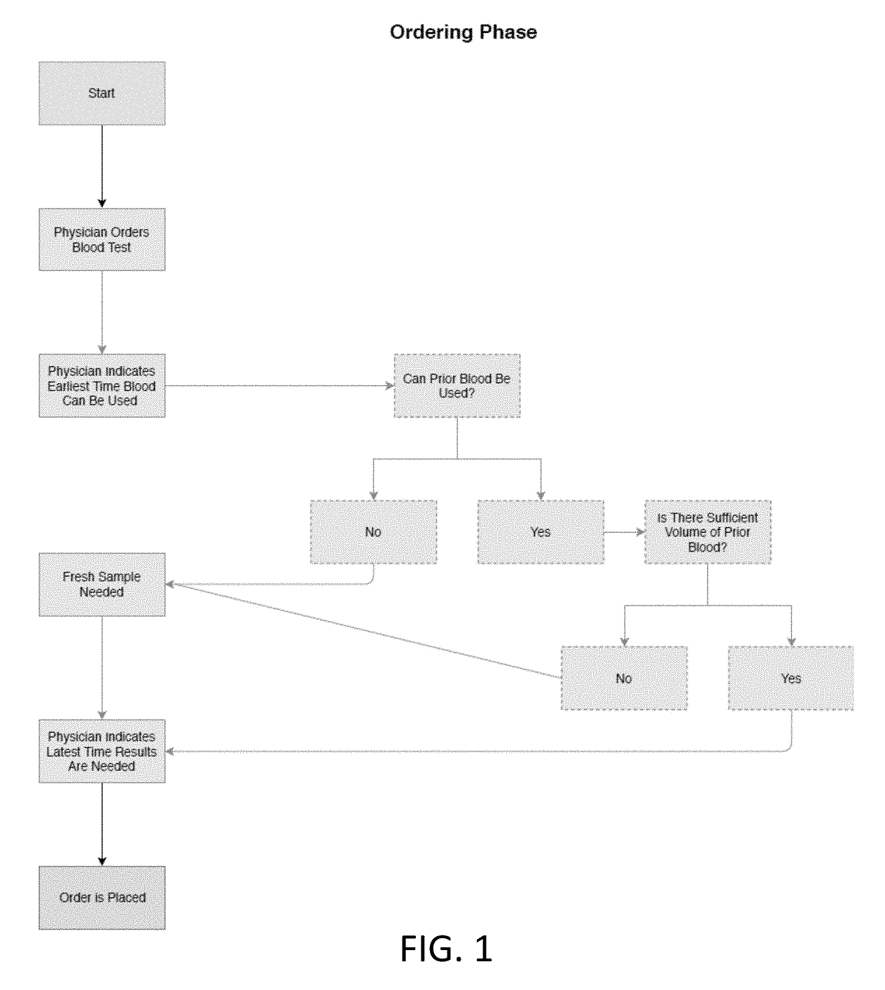

Referring the FIG. 1, using the provider user interface, the health care provider inputs information related to a request for a laboratory blood test. 10 The information may be collected via an interactive graphical user interface (“GUI”). The information may include: an identifier for the specific patient; an identifier for the health care provider; the blood test type; the time of the order, the time the test results are required, and the time the provider plans to review the test results. The order may include other information, including a diagnosis for the patient's condition, and identifiers for other health care providers who will review the test results. For example, the request may indicate the earliest time a blood sample can be used. 12 Additional queries may include provider information including the earliest time blood can be used and the latest time that the provider must have the laboratory results available.

An embodiment of the invention may include a test profile database, containing electronically stored information concerning blood test use. The database is available to the central computer processor. The database may include data regarding the relationships between blood tests, diagnosis, and related or commonly requested secondary and/or additional tests. The test profile database may include the ability to compile data based on patterns of testing procedures, including the past patterns of provider requests for blood tests and secondary and/or additional tests. The test profile database may include information concerning the cost and reimbursement requirements for tests.

An embodiment of the invention may include a schedule database. Schedule database may collect information relating to the availability and status of one or more of the health care providers, patients, phlebotomy staff, and laboratory staff. For example, the schedule database may identify times where a patient is not available for a venipuncture procedure, such as when the patient is asleep or in surgery. In some situations, a patient may be in radiology or in physical therapy and unavailable for a blood draw. Physical therapy appointments, for example, often are prescheduled and in the patient's charts/schedules. The central computer processor will notify/update the expected time of a venipuncture in the patient's EMR. This will appear on the patient's chart as a prescheduled appointment/procedure. The schedule database may contain information concerning the status of a healthcare provider, indicating when the provider will be available to review test results. The schedule database may include information regarding phlebotomy and laboratory staff, indicating when appropriate staff is available to conduct a particular venipuncture procedure. The schedule database may contain information relating to the length of time necessary for a phlebotomist to travel to the patient to draw the sample and return the sample to the lab, as well as the time necessary to process the sample (complete the test) and post the results The schedule database may provide schedule data to the central computer processor via an electronic communication connection or network.

In one embodiment of the invention, the central computer processor may receive data from the provider user interface and obtain relevant data from the test profile database and schedule database. Based on one or more data available, the central computer processor generates blood test parameters. The blood test parameters may include: The time a sample may be collected, the conditions required for the ordered tests, the latest time a sample can be collected, and the amount of sample to be collected.

The central computer processor will use the information received from the data from the provider user interface and obtain relevant data from the test profile database and schedule database to establish parameters for the sample collection. For example, the data may include the specific test ordered, the name or identifier of the ordering physician, and the name or identifier of the patient. When a health professional orders a test, the time of the order is logged. The time at which a clinician draws blood from the patient also is logged. When the laboratory completes the test and posts the result of that test, that time is logged. When the physician reviews the result of the test, that time is logged.

From this data, a number of test parameters may be computed. For example, time that it takes from Order to Draw is calculated by the difference between the Order Time and Draw Time. The time that it takes from Draw to Results is calculated by the difference between the Draw Time and Results Time. The time that it takes from Results to Review is calculated by the difference between the Results Time and Review Time.

The frequency and percentages of each test ordered is calculated, and each tests' statistics; i.e. how long each test takes to process, collect, as well as the frequency of any follow-up tests, may be continuously logged and stored.

In one embodiment of the invention, the central computer processor may generate one or more reports based upon information collected concerning blood laboratory procedures. The report may include feedback from the clinical pathology department on the efficiency of his/her team and processes, as well as optimal staffing requirements. For example, the report may give averages for all data collected and compare them to previous weeks, months, and years. It may also compare data to corresponding data collected at other hospitals nationwide. The report may include data allowing a hospital or healthcare Center to assess the efficiency of their blood laboratory operations relative to their past performance and national benchmarks including how many and what types of tests were ordered and specific ordering providers. The report may include information of interest to other departments such as hospitalists, internal medicine, and surgery regarding which tests may be overutilized relative to national benchmark averages for related diagnoses and specific ordering providers. The reports may contain information required or requested by government agencies, including the Centers for Medicare & Medicaid Services (CMS), to aid them in developing updated appropriate Medicare Severity-Diagnosis Related Group (MS-DRG) reimbursements. The report may contain information useful to consultants retained to evaluate and optimize laboratory procedures by quantitatively documenting improvement in patient care and healthcare cost reduction.

In one embodiment of the invention, the system of the invention may collect and report information that can be used to monitor the operations of the system. Information that will be on the reports include: Number of specific blood tests ordered (which can be analyzed by patient, diagnosis, or ordering provider); number of needle sticks per patient per day; time difference from order to draw to results; time difference from draw to results; time difference from results to review; time difference from physician indicated time to actual review time; and hospital bed census. The reports and information may provide the user with guidance in optimizing the efficiencies related to the data.

Based on the data from the provider order user interface and obtain relevant data from the test profile database and schedule database, the central computer processor determines a schedule for receiving a patient blood sample, including the patient's identifying information, the patient's location, the type of draw required, the amount of blood to be samples, the schedule for test required, the schedule for predicted tests, and other parameters. The schedule minimizes the frequency of blood draws, reducing cost and minimizing risks to phlebotomists and patients

In practice, one embodiment of the apparatus and system of the invention will operate according to the following example. First, a physician or other healthcare provider orders a laboratory blood test for a patient. 10 For example, a physician may order a CBC blood test for patient ‘A’ at 10:00 AM, using a provider user interface. The physician indicates that she needs the results no later than 3:00 PM. She also may indicate that the earliest time a sample of prior blood could be used was 08:00 AM. 12

As soon as this order is submitted, the central computer processor will determine if prior blood can be used. 14 By querying the test database, the processor determines that blood samples from 8:00 AM and beyond are suitable for this test. If it finds a suitable prior blood sample, it will then check to determine to see if there is enough blood in storage to run the test, based on the test database information regarding the amount of blood collected at 8:00 AM and the amount of blood required to perform the CBC test. 16 If the central computer processor determines that the blood in storage cannot be used, it will determine the parameters for a new blood draw based on the time required to collect a sample, process the sample, and the time and lab results are required. 18 The central computer processor may query the scheduler database to determine the availability of phlebotomy staff, the patient schedule, and the schedule of the ordering physician and other healthcare providers who will have access to the test results. Once this is all determined, the central computer processor will postpone the test as long as possible to allow other orders to accumulate before the results are needed at 1500. 20 Based on these parameters, the central computer processor initiates an order for a blood draw. 22

In the foregoing example the apparatus and system of the invention may accommodate other blood laboratory request by other providers. For example, if at 1:00 PM, a different provider orders Fibrinogen and Hematocrit blood tests for the same patient, and indicates that he requires the test results no later than 4:00 PM, the central computer processor will combine these two additional test orders to the existing order in Patient A's queue. The central computer processor will determine the limiting factor among the group of blood tests based on factors such as processing times and the times that the doctors requested the results be available to them.

Continuing with the example above, once one or more test orders are placed, the central computer processor will query the test database to predict whether additional tests are likely to be ordered within a defined sample window. The prediction may be based upon a particular health care providers past practice of ordering follow-up tests, or historical data concerning the likelihood that additional follow-up tests will be required based on the results from a given order test. If the central computer processor predicts that an additional laboratory blood test is likely, it will query the test database to determine the amount of blood required to perform the test and reserve a fraction of the scheduled blood draw to perform that test.

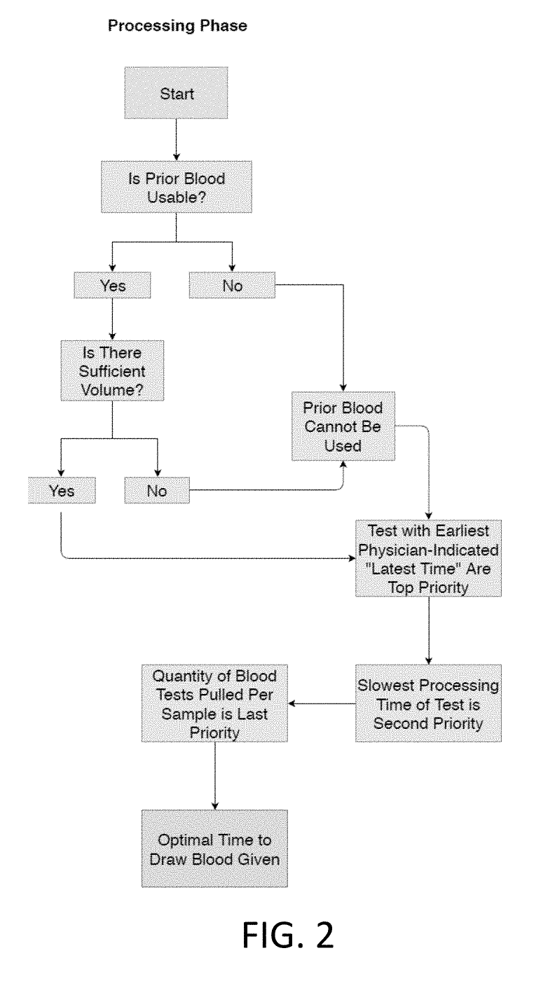

Referring to FIG. 2, in some cases subsequent test orders may be performed using a sample reserve fraction collected based on predicted future tests. In that case, the central computer processor will determine whether the prior blood sample is usable, based upon the collection time and collection conditions. 24 if the sample reserve fraction has sufficient volume 26, then the test will be performed according to a priority based upon the provider indicated “latest-time” for the test. 28 If the reserve sample fraction is not of sufficient volume, then the prior sample cannot be used to perform the test. 30. Laboratory tests are performed according to priority, with the slowest processing time a second priority. 32 If the reserve sample fraction is exceeded, then an additional blood draw order is placed for a defined quantity of sample, 34 and the blood draw is scheduled based upon the optimal parameters.

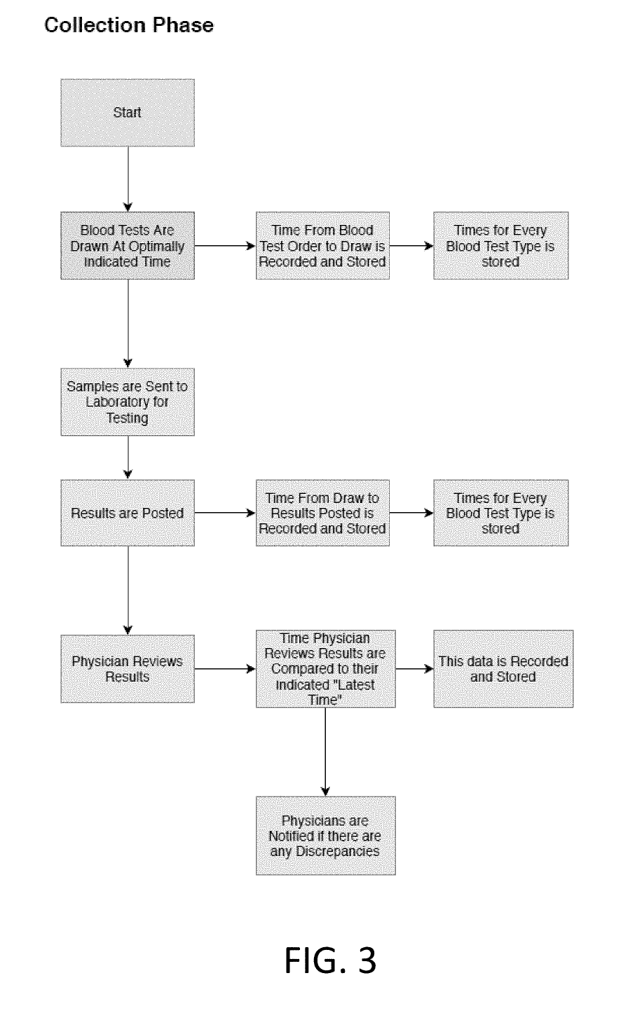

Continuing with the example above, if no other tests orders are submitted, the central computer processor will generate a number of parameters for the blood draw, including the optimal blood draw time, the amount of blood required for the ordered tests, and in the additional amount of blood to be stored for predicted tests, ensuring that all test results reach the healthcare providers at their requested times. As shown in FIG. 3, samples are collected based on parameters including the times that test results are required. 36 In some embodiments, the time from blood test order to blood draw is recorded and stored. 38 after collection, samples are sent to the laboratory for testing. 40. Following completion of tests, test results are provided to the requesting provider, and in some embodiments may be posted to the EMR. 42 In some embodiments the time between the blood draw and the posted results is recorded and stored, and the times for each blood test type is stored. 44 After posting of results, the provider reviews the results 46 and the time that the results are reviewed is stored and compared to the “latest time” on the test order. 48 The differential between the “latest time” on the test order and the providers review of results is recorded and stored. 50 The provider is notified if any discrepancies exist between the ordered time and the provider's actual review of test results. 52

Blood is collected in certain color-coded tubes depending on what test is needed. The following table indicates the most common tube top colors, volume of tubes, and commonly ordered tests collected in each tube.

| TUBE TOP | TUBE | |
|---|---|---|
| COLOR | VOLUME | TESTS PERFROMED |
| LIGHT BLUE | 5 mL | Prothrombin Time (PT), Partial |
| | | Thromboplastin, Time (PTT), |
| | | Thrombin Time (TT), Fibrinogen, |
| | | Fibrin degradation products (FDP), |
| | | D-dimer |
| RED | 7 mL | ABO/D type aka ABO/Rh Testing, |
| | | Antibody Screen AKA Indirect |
| | | Antiglobulin Test (IAT), Type and |
| | | Screen (T&S), Crossmatch or Type |
| | | and Cross (T&C), Rh Immune |
| | | Globulin AKA Rhogam, Antibody |
| | | Titer, Antigen Typing |
| GREEN | 7 mL | Plasma determinations |
| LAVENDER | 5 mL | Hemoglobin, Hematocrit, White |
| | | blood cell (WBC) count, Red blood |
| | | cell (RBC) count including MCV, |
| | | MCH, and MCHC, Platelet Count, |
| | | Differential, Complete Blood |
| | | Count, Reticulocyte count, |
| | | Eosinophil count |
| GRAY TOP | 7 mL | Glucose Levels, Blood Alcohol |
| | | Levels, Lactate, Bicarbonate |

Once blood is collected, a sample is sent to the laboratory for testing. Once test is concluded, the results are posted, and tubes are stored in a refrigerator for a hospital-specified time. For example, some hospitals store blood in tubes for 8 hours, while others do not store at all. In some embodiments of the invention, the laboratory or health facility will have a storage facility that stores and sorts blood by patient. The storage facility would utilize the Internet of Things (IoT) and have real time data of types of blood, tubes, and volume of blood in tube. Having sensors on blood collection tubes and storage refrigerator will indicate exact volume (in mL) of blood in each tube. With this information, the computer processor will know the age and volume of samples in storage. Physicians will then have the necessary real-time data when ordering tests that can be conducted using prior blood. By using IoT, we will also be able to track where phlebotomists and patients are located in the hospital to optimize scheduling.

Laboratory tests require a minimum volume of blood to conduct a test. Examples are: CBC (1.5 ml minimum), Blood Glucose (0.25 mL minimum), Fibrinogen (0.5 mL minimum), Lactate (1.0 mL), etc.

This information is imperative for knowing if/when tests can be consolidated in a single tube. For example: Two samples are ordered, and each test (Blood glucose and Lactate) require a Gray top tube. One Gray top tube has a capacity to hold 7 mL of blood. The blood glucose test requires a blood sample of 0.5 mL, and the Lactate test requires a blood sample of 1.0 mL. it can be determined from the tube top colors that one gray top tube is sufficient for the combined 1.5 mL sample from both tests.

Knowing and storing data regarding how much blood each test requires is also used when predicting future tests. If data on blood tests is congregated and stored, the central computer processor is able to offer information on frequently ordered tests after specific (normal/abnormal) test results. For instance, a physician can be notified that “In the event of an abnormal ‘A’ test, ‘B’ test is ordered X % of the time”, and can provide information on the percentages of an abnormal test coming from patients with similar conditions and previous test results and even offer differential considerations for the patient's condition. With this knowledge, a physician may wish to order an additional “future” test based on these findings—as opposed to waiting to see the results, and then order an additional test/stick. Having a congregation of continuous real-time data on how much blood is needed per test, if there is sufficient volume in the collection tube to add this test on, it will prevent unnecessary needle sticks, unnecessary risks that come along with procedures, supplies, time, and money for the hospital.

After placing the laboratory order, the physician will also be notified of an estimated time he/she should expect the results to be posted. Based on continuous real-time congregated data, the processing times of each test are known. These times can vary depending on the time of day, day of week, month, year, etc. For example, on a certain day the turnaround time for a CBC (time from order to results posted) may be 2 hours 3 minutes 12 seconds. On another day, the turnaround time for a CBC may be 1 hour 17 minutes 39 seconds. This information, congregated over time, can determine trends, benchmarks, and other useful information for hospitals—in terms of staffing, ordering of supplies, monitoring physician trends, etc.

In one embodiment of the invention, the central computer processor will distribute test results via the patient's electronic medical record. Once the results are posted, the central computer processor will monitor when the doctor reviews the test. If tests are not reviewed within an hour of the indicated time, the physician will be notified.

When a physician is logged on to their patient in EMR, the central computer processor will offer information on frequently ordered tests after posting an abnormal test result. For instance, “In the event of an abnormal ‘A’ test, ‘B’ test is ordered X % of the time”, and can provide information on the percentages of an abnormal test coming from patients with similar conditions and previous test results and even offer differential considerations for the patient's condition.

The preceding description is exemplary rather than limiting in nature. Variations and modifications to the disclosed examples may become apparent to those skilled in the art that do not necessarily depart from the essence of this disclosure. The scope of legal protection given to this disclosure can only be determined by studying the following claims.

What is claimed is:

1. A system for collection, storage, and processing of laboratory tests comprising:
a central computer processor;
a provider user interface operably connected to said computer processor;
a test profile database operably connected to said computer processor;
a schedule database operably connected to said computer processor; and
A tissue sample storage facility;
whereby the computer processor is adapted to receive tissue sample orders from the provider user interface, test profiles from the test profile database, and patient and provider schedules from the schedule database;
the computer processor is adapted to calculate test parameters; and
the computer processor sends a tissue sample according to the test parameters comprising a reserve sample fraction.

2. The system of claim 1 wherein the central processor, provider user interface, test profile database, and schedule database are operably connected by an electronic computer network.

3. The system of claim 1 wherein the tissue sample is human tissue.

4. The system of claim 1 wherein the tissue sample is human blood.

5. The system of claim 1 further comprising a laboratory test results interface to communicate data to a patient electronic medical record.

6. The system of claim 1 wherein the provider user interface comprises parameters for the earliest time a sample can be used and the latest time the provider must receive the results.

7. A method for collection and processing laboratory blood samples comprising:
receiving an order from a provider user interface;
receiving first data from a test profile database;
receiving second data from schedule database;
predicting the need for secondary tests;
generating parameters for a sample collection;
obtaining a sample, comprising a reserve sample fraction, according to the parameters; and
storing the reserve sample fraction of said sample.

8. The method of claim 7 further comprising exporting test results to the patient electronic medical record.

9. The method of claim 7 further comprising the steps of receiving from the provider user interface data regarding the earliest time a sample can be used and the latest time that the provider must receive the results.

10. The method of claim 9 wherein the sample is human blood.
